(19) United States
(12) Patent Application Publication
(10) Pub. No.: US 2026/0009700 A1
(43) Pub. Date: Jan. 8, 2026

(54) AUTOMATIC PRESSURE-RETAINING SAMPLING APPARATUS AND METHOD FOR DEEP-SEA SPECIFIC LAYER SEAWATER

(71) Applicants: GUANGDONG UNIVERSITY OF TECHNOLOGY, Guangdong (CN); GUANGDONG LABORATORY OF SOUTHERN OCEAN SCIENCE AND ENGINEERING (GUANGZHOU), Guangdong (CN)

(72) Inventors: Jingchun FENG, Guangdong (CN); Si ZHANG, Guangdong (CN); Jianzhen LIANG, Guangdong (CN); Yan XIE, Guangdong (CN)

(73) Assignees: GUANGDONG UNIVERSITY OF TECHNOLOGY, Guangdong (CN); GUANGDONG LABORATORY OF SOUTHERN OCEAN SCIENCE AND ENGINEERING (GUANGZHOU), Guangdong (CN)

(21) Appl. No.: 19/260,305

(22) Filed: Jul. 4, 2025

(30) Foreign Application Priority Data
Jul. 5, 2024 (CN) 202410896783.X

(51) Int. Cl. G01N 1/10; G01N 33/18

(52) U.S. Cl. CPC G01N 1/10; G01N 33/1886; G01N 2001/1031; G01N 2001/1062

(57) ABSTRACT

The present invention discloses an automatic pressure-retaining sampling apparatus and method for deep-sea specific layer seawater, relating to the technical field of deep-sea sampling. The sampling apparatus includes a rotation unit configured to drive sampling valves in a multi-sequence sampling filter unit to be opened and closed; the multi-sequence sampling filter unit configured to sample seawater and stabilize plankton; an injection unit configured to inject seawater or stabilization solution into the multi-sequence sampling filter unit; a pressurization unit configured to adjust a pressure of the multi-sequence sampling filter unit; a layer identification unit configured to automatically identify deep-sea target layers; a control unit configured to receive data transmitted from other units and control other units; and an outer frame configured to support other units. According to the present invention, through the coordinated operation of various units, a specific seawater layer can be automatically selected.

### CROSS REFERENCE TO THE RELATED APPLICATIONS

This application claims the priority benefit of China application serial no. 202410896783.X, filed on Jul. 5, 2024. The entirety of the above-mentioned patent applications is hereby incorporated by reference herein and made a part of this specification.

### BACKGROUND

### Technical Field

The present invention relates to the technical field of deep-sea sampling, and more specifically, to an automatic pressure-retaining sampling apparatus and method for deep-sea specific layer seawater.

### Description of Related Art

The ocean occupies 71% of the Earth's surface area and is the largest habitat for Earth's organisms. The deep sea generally refers to regions with water depths exceeding 1000 meters and is considered an extreme environment featuring darkness, high hydrostatic pressure, low/high temperatures, and oligotrophic conditions. In the entire vertical marine environment, due to changes in conditions such as light, temperature, salinity, and pressure, the seawater profile is divided into various specific seawater layers, including the euphotic layer, twilight layer, and aphotic layer. Currently, most piezophilic plankton in the deep-sea environment have not been acquired with integrity being preserved or isolated for cultivation, and their physiological and metabolic mechanisms can only be inferred based on genomics and environmental response studies, making it difficult to accurately quantify the specific physiological and metabolic characteristics of a living organism. Additionally, due to the lack of data on environmental changes and biological responses between specific vertical layers of deep-sea seawater, it is difficult to quantify the mysterious carbon cycle processes in the vertical direction of the deep-sea environment, hindering systematic understanding of ocean carbon sink processes. Therefore, it is necessary to obtain integrity-preserving seawater samples from specific vertical layers of the deep-sea environment for conducting integrity-preserving cultivation of marine plankton and identifying metabolic reactions. Obtaining highly active microorganisms is the foundation for in-depth scientific research and resource development applications, as well as an important support for further understanding deep-sea microorganisms and their ecological processes.

For sample acquisition in deep-sea multi-layer environments, existing technologies mainly obtain environmental samples under normal pressure conditions or perform in-situ filtration and stabilization of plankton, while obtaining pressure-retaining seawater samples or highly active plankton in high-pressure environments is quite difficult. Moreover, in the deep-sea environment, the effect of seawater sampling is difficult to be evaluated, and the sampling effect can be evaluated only after the sampling apparatus is retrieved to the ship. When the sampled seawater does not meet the sampling requirements, the sampling apparatus must be lowered again for resampling, resulting in a low sampling efficiency and large sampling errors. Overall, existing sampling apparatuses have issues such as complex operation, fixed seawater sampling layers, difficulty in evaluating the sampling effect, and inability to automatically perform secondary sampling for failed samples, posing significant challenges to efficiently obtaining seawater integrity-preserving samples from deep-sea specific layer. Given this situation, there is an urgent need to provide a sampling apparatus capable of automatically selecting a specific seawater layer, automatically evaluating a sampling effect, and automatically performing secondary sampling for failed samples, to provide an important foundation for improving the understanding of deep-sea in-situ scientific principles and the development of biological resources.

### SUMMARY

To overcome the shortcomings of the existing deep-sea sampling apparatus during sampling of specific seawater layers, such as complex operation, inability to automatically select a specific seawater layer, difficulty in evaluating a sampling effect, and inability to automatically perform secondary sampling for failed samples, the present invention provides an automatic pressure-retaining sampling apparatus and method for deep-sea specific layer seawater.

To address the above technical problems, the technical solution of the present invention is as follows:

The present invention provides an automatic pressure-retaining sampling apparatus for deep-sea specific layer seawater, including an outer frame, a rotation unit, a multi-sequence sampling filter unit, an injection unit, a pressurization unit, a layer identification unit, and a control unit.

The multi-sequence sampling filter unit is disposed in the outer frame and includes a plurality of sampling filter modules, and each of the sampling filter modules includes a sampling valve, a sampling kettle, an exhaust solenoid valve, a pressure sensor, a filter valve, a filter kettle, and a flow rate meter; a water outlet end of the sampling valve is connected to one end of the sampling kettle, and the other end of the sampling kettle is provided with the pressure sensor and the exhaust solenoid valve; the water outlet end of the sampling valve is further connected to a water inlet end of the filter valve, a water outlet end of the filter valve is connected to one end of the filter kettle, and the other end of the filter kettle is connected to the flow rate meter; and the plurality of sampling valves are circumferentially distributed at a top of the outer frame, and control ends of all the sampling valves face the rotation unit.

The injection unit is disposed in the outer frame and includes a water pump, a stabilization solution injection pump, and a stabilization solution storage kettle; where one end of the water pump is suspended, and the other end of the water pump is connected to a water inlet end of the sampling valve of each of the sampling filter modules; and one end of the stabilization solution injection pump is connected to the stabilization solution storage kettle, and the other end of the stabilization solution injection pump is connected to one end of the filter kettle of each of the sampling filter modules.

The rotation unit is disposed at a center of the top of the outer frame, and an end of the rotation unit abuts against the control end of the sampling valve.

The pressurization unit is disposed outside the outer frame, and an output end of the pressurization unit is connected to the other end of the sampling kettle of each of the sampling filter modules.

The layer identification unit is disposed at a bottom of the outer frame, and a data output end of the layer identification unit is connected to a data input end of the control unit.

The control unit is disposed in the outer frame, the data input end of the control unit is further connected to data output ends of the pressure sensor and the flow rate meter, and a control end of the control unit is connected to control ends of the rotation unit, the pressurization unit, and the exhaust solenoid valve.

With the arrangement of the layer identification unit, actual environmental characteristics of the seawater profile are automatically identified during the lowering process. The control unit determines a plurality of target layers and calculates corresponding pre-charged pressure values and controls the pressurization unit to sequentially inject high-pressure nitrogen gas into the corresponding sampling kettles. When the sampling apparatus reaches the depth of a target layer during the lifting process, the rotation unit rotates clockwise to open the corresponding sampling valve in the multi-sequence sampling filter unit and the rotation unit stop rotating when the corresponding sampling valve is opened, while the control unit activates the water pump and opens the filter valve to start sampling. After a period of sampling, the rotation unit rotates clockwise to close the sampling valve and the rotation unit stop rotating when the sampling valve is closed, and the control unit deactivates the water pump. The pressure sensor and the flow rate meter respectively monitor a real-time pressure of the sampling kettle and a real-time flow rate of the filter kettle in real time. The control unit determines whether the real-time pressure and the real-time flow rate reach preset thresholds. If both thresholds are reached, the stabilization solution injection pump is activated to stabilize the plankton in the filter kettle. Otherwise, the rotation unit rotates counterclockwise to reopen the sampling valve and the rotation unit stop rotating when the sampling valve is reopened, and the pressurization unit is controlled to inject a pressure greater than the in-situ pressure of the target layer into the sampling kettle, discharging the seawater in the sampling kettle through the reopened sampling valve. The pressure sensor monitors the pressure in the sampling kettle in real time, and when the real-time pressure in the sampling kettle reaches a preset pressure, it indicates that the seawater has been completely discharged. Then, the exhaust solenoid valve is controlled to be opened, discharging the gas until a value of pressure in the sampling kettle reaching the pre-charged value. The water pump is then activated again for secondary sampling until the seawater sampling at the target layer is successful. The sampling apparatus is lifted again to the next target layer, and the above operations are repeated until the seawater sampling for all target layers is completed.

Preferably, the sampling kettle includes a first kettle body, a piston, and a rubber ring.

A sidewall of the piston is provided with a groove, and the rubber ring is sleeved in the groove of the piston.

The piston is disposed in the first kettle body and divides the first kettle body into a gas chamber and a liquid chamber.

one end of the liquid chamber of the first kettle body is connected to the water outlet end of the sampling valve, and one end of the gas chamber of the first kettle body is provided with the pressure sensor and the exhaust solenoid valve.

The sampling kettle is made of high-pressure-resistant metal material and the internally mounted piston is a pressure-resistant piston. The sidewall is of a groove structure for mounting the rubber ring. The first kettle body is divided by the piston into the gas chamber and the liquid chamber, where the liquid chamber is used for receiving seawater samples, and the gas chamber is used for receiving high-pressure nitrogen gas. The pressure sensor is configured to measure the internal pressure value of the sampling kettle in real time and transmit it to the control unit, and the exhaust solenoid valve is configured to discharge high-pressure nitrogen gas exceeding the pre-charged pressure value during secondary sampling.

Preferably, the pressurization unit includes a nitrogen gas cylinder, a cylinder holder, a pressure regulator, and a gas injection solenoid valve.

The cylinder holder is disposed outside the outer frame, and the nitrogen gas cylinder is disposed in the cylinder holder.

A gas outlet of the nitrogen gas cylinder is connected to one end of the pressure regulator, the other end of the pressure regulator is connected to one end of the gas injection solenoid valve, and the other end of the gas injection solenoid valve is connected to one end of the gas chamber of each of the sampling kettles.

The control end of the control unit is connected to control ends of the pressure regulator and the gas injection solenoid valve.

The nitrogen gas cylinder is sequentially connected to the gas chamber of the sampling kettle via the pressure regulator and the gas injection solenoid valve. The pressure regulator is configured to automatically adjust the pressure value of the injected nitrogen gas. The cylinder holder is configured to secure the nitrogen gas cylinder to prevent the cylinder from tipping over during ship operations or drifting underwater due to ocean currents and buoyancy. The cylinder holder includes three layers of railings, with latches provided on the railings on the same side of the three layers, all of which can be opened outward, facilitating the transfer of the nitrogen gas cylinder into the cylinder holder and secure locking.

Preferably, the rotation unit includes a rotation motor, a gear disc, and a double-end ejector rod.

The gear disc is disposed at the center of the top of the outer frame.

The double-end ejector rod includes a long end portion and a short end portion, the short end portion of the double-end ejector rod abuts against a tooth surface of the gear disc, and the long end portion of the double-end ejector rod abuts against the control end of the sampling valve.

An output end of the rotation motor is connected to the double-end ejector rod to drive the double-end ejector rod to rotate, and a control end of the rotation motor is connected to the control end of the control unit.

The gear disc is disposed at the center of the top of the outer frame, and the double-end ejector rod is driven by the rotation motor. After rotation, the long end portion of the double-end ejector rod contacts a round ejector pin of the sampling valve, triggering the opening and closing of the sampling valve. The short end portion of the double-end ejector rod has a spring-loaded pin, and the spring-loaded pin, after rotation, engages in the tooth groove of the gear disc for fixation. The tooth grooves of the gear disc correspond to the sampling valves to achieve accurate opening and closing of the sampling valves.

Preferably, the layer identification unit includes a depth sensor, a temperature sensor, and a salinity sensor.

The depth sensor, the temperature sensor, and the salinity sensor are all disposed at the bottom of the outer frame, and data output ends of the depth sensor, the temperature sensor, and the salinity sensor are connected to the data input end of the control unit.

The layer identification unit may be a conductivity-temperature-depth (CTD) instrument to measure the conductivity, temperature, and depth parameters of the ocean water body.

Preferably, the injection unit further includes a plurality of injection valves.

The other end of the stabilization solution injection pump is connected to one end of each of the injection valves, and the other end of each of the injection valves is connected to one end of the filter kettle of one sampling filter module correspondingly.

Preferably, the filter kettle includes a second kettle body, a filter screen frame, a filter membrane, and a filter screen.

The filter screen frame is disposed in the second kettle body, the filter screen is wrapped around the filter screen frame, and the filter membrane is disposed on the filter screen.

One end of the second kettle body is connected to the water outlet end of the filter valve and the other end of the injection valve, and the other end of the second kettle body is connected to the flow rate meter.

The filter screen frame has multiple holes with a diameter of 5 mm, the filter screen has holes with a diameter of smaller than 1 mm, and the filter membrane has a pore size of 0.22 μm. The filter screen is configured to support the filter membrane. The flow rate meter is configured to measure the flow rate of filtered in-situ seawater and calculate the volume of filtered in-situ seawater.

Preferably, the stabilization solution storage kettle contains RNAlater stabilization solution.

The present invention further provides an automatic pressure-retaining sampling method for deep-sea specific layer seawater, applied to the above sampling apparatus, and including:
S1: evacuating each sampling kettle to a vacuum state and lowering the sampling apparatus to a seabed; and during a lowering process, measuring, by a layer identification unit, a real-time temperature, a real-time salinity, and a real-time depth of the seawater, and sending the real-time temperature, the real-time salinity, and the real-time depth to a control unit;
S2: identifying, by the control unit, a plurality of target layers, calculating corresponding pre-charged pressure values based on the real-time temperature, the real-time salinity, and the real-time depth of the seawater, controlling a pressure regulator to make adjustment to the corresponding pre-charged pressure values sequentially, controlling a gas injection solenoid valve to be opened, and enabling a nitrogen gas cylinder to inject high-pressure nitrogen gas into corresponding sampling kettles sequentially; monitoring, by a pressure sensor, a current pressure of the sampling kettle in real time; and sending a lifting signal to the control unit when the current pressure of the sampling kettle reaches the corresponding pre-charged pressure value;
S3: lifting the sampling apparatus to one target layer; and controlling, by the control unit, a rotation motor to rotate clockwise to drive a long end portion of a double-end ejector rod to open a corresponding sampling valve and the rotation motor to stop rotate when the corresponding sampling valve is opened, controlling a water pump to be activated, opening a corresponding filter valve to perform seawater sampling, after a first preset time, deactivating the water pump, and controlling the rotation motor to rotate clockwise to drive the long end portion of the double-end ejector rod to close the corresponding sampling valve and the rotation motor to stop rotate when the corresponding sampling valve is closed;
S4: monitoring, by the pressure sensor and a flow rate meter, the current pressure of the sampling kettle and a real-time flow rate of a filter kettle in real time respectively, and sending the current pressure and the real-time flow rate to the control unit; and performing step S7 when the current pressure of the sampling kettle exceeds a preset pressure threshold and the real-time flow rate of the filter kettle exceeds a preset flow rate threshold; otherwise, performing step S5;
S5: controlling, by the control unit, the rotation motor to rotate counterclockwise to drive the long end portion of the double-end ejector rod to reopen the sampling valve and the rotation motor to stop rotate when the sampling valve is reopened, closing the filter valve, and controlling the nitrogen gas cylinder to continuously inject high-pressure nitrogen gas into the sampling kettle, so as to discharge the seawater in the sampling kettle via the reopened sampling valve until the sampling kettle reaches a second pressure;
S6: opening an exhaust solenoid valve and controlling the water pump to be reactivated to perform seawater sampling; and when a cumulative flow rate of the flow rate meter reaches a preset cumulative flow rate threshold, controlling the water pump and the exhaust solenoid valve to be deactivated, and controlling the rotation motor to rotate clockwise to drive the long end portion of the double-end ejector rod to close the sampling valve and the rotation motor to stop rotate when the sampling valve is closed; and returning to step S4;
S7: controlling a stabilization solution injection pump to activate, to inject RNAlater stabilization solution from a stabilization solution storage kettle into a corresponding filter kettle, so as to stabilize plankton in a filter membrane; and deactivating the stabilization solution injection pump after a second preset time; and
S8: determining whether seawater sampling for all target layers is completed; and if the seawater sampling for all target layers is not completed, lifting the sampling apparatus to a next target water sampling layer and repeating steps S3 to S7; otherwise, terminating the seawater sampling.

Preferably, the identifying, by the control unit, the plurality of target layers and calculating the corresponding pre-charged pressure values based on the real-time temperature, the real-time salinity, and the real-time depth of the seawater includes:

Using a plurality of seawater layers, each having a variation value of the real-time temperature or the real-time salinity of the seawater exceeding a preset variation threshold, as the target layers, and calculating the corresponding pre-charged pressure values of the target layers based on the corresponding real-time depths:

$${N}_{i} = \frac{( V - {V}_{L} ) {D}_{i}}{100 \times V}$$

where Nᵢ represents a pre-charged pressure value corresponding to an i-th target layer, Dᵢ represents a real-time depth of the i-th target layer, V represents a volume of the sampling kettle, and V_{L} represents an expected volume after in-situ pressure compression of the sampling kettle at the i-th target layer.

As compared with the prior art, the technical solution of the present invention has the following beneficial effects:

According to the automatic pressure-retaining sampling apparatus and method for deep-sea specific layer seawater provided by the present invention, with the arrangement of the layer identification unit, actual environmental characteristics of the seawater profile are automatically identified during the lowering process. The control unit determines a plurality of target layers and calculates corresponding pre-charged pressure values and controls the pressurization unit to sequentially inject high-pressure nitrogen gas into the corresponding sampling kettles. When the sampling apparatus reaches the depth of a target layer during the lifting process, the rotation unit rotates clockwise to open the corresponding sampling valve in the multi-sequence sampling filter unit and the rotation unit stop rotating when the corresponding sampling valve is opened, while the control unit activates the water pump and opens the filter valve to start sampling. After a period of sampling, the rotation unit rotates clockwise to close the sampling valve and the rotation unit stop rotating when the sampling valve is closed, and the control unit deactivates the water pump. The pressure sensor and the flow rate meter respectively monitor a real-time pressure of the sampling kettle and a real-time flow rate of the filter kettle in real time. The control unit determines whether the real-time pressure and the real-time flow rate reach preset thresholds. If both the real-time pressure and the real-time flow rate reach preset thresholds, the stabilization solution injection pump is activated to stabilize the plankton in the filter kettle. Otherwise, the rotation unit rotates counterclockwise to reopen the sampling valve and the rotation unit stop rotating when the sampling valve is reopened, and the pressurization unit is controlled to inject a pressure greater than the in-situ pressure of the target layer into the sampling kettle, discharging the seawater in the sampling kettle through the reopened sampling valve. The pressure sensor monitors the pressure in the sampling kettle in real time, and when the real-time pressure in the sampling kettle reaches a preset pressure, it indicates that the seawater has been completely discharged. Then, the exhaust solenoid valve is controlled to open, discharging the gas until a value of pressure in the sampling kettle reaching the pre-charged value. The water pump is then activated again for secondary sampling until the seawater sampling at the target layer is successful. The sampling apparatus is lifted again to the next target layer, and the above operations are repeated until the seawater sampling for all target layers is completed. According to the present invention, a specific seawater layer can be automatically selected, a sampling effect can be autonomously evaluated, and a failed sample can be automatically discharged and followed by secondary sampling, allowing for a high sampling efficiency and a good sampling effect.

### BRIEF DESCRIPTION OF THE DRAWINGS

FIG. 1 is a schematic structural diagram of an automatic pressure-retaining sampling apparatus for deep-sea specific layer seawater according to Embodiment 1.

FIG. 2 is a schematic structural diagram of an automatic pressure-retaining sampling apparatus for deep-sea specific layer seawater according to Embodiment 2.

FIG. 3 is a schematic structural diagram of a sampling kettle according to Embodiment 2.

FIG. 4 is a schematic structural diagram of a filter kettle according to Embodiment 2.

FIG. 5 is a schematic structural diagram of a rotation unit according to Embodiment 2.

FIG. 6A and FIG. 6B are a flowchart of an automatic pressure-retaining sampling method for deep-sea specific layer seawater according to Embodiment 3.

### DESCRIPTION OF THE EMBODIMENTS

The drawings are for illustrative purposes only and should not be construed as limiting this patent.

To better illustrate this embodiment, certain components in the drawings may be omitted, enlarged, or reduced, and do not represent the actual product dimensions.

For those skilled in the art, it is understandable that certain well-known structures in the drawings and their descriptions may be omitted.

The technical solution of the present invention is further described below with reference to the drawings and embodiments.

### Embodiment 1

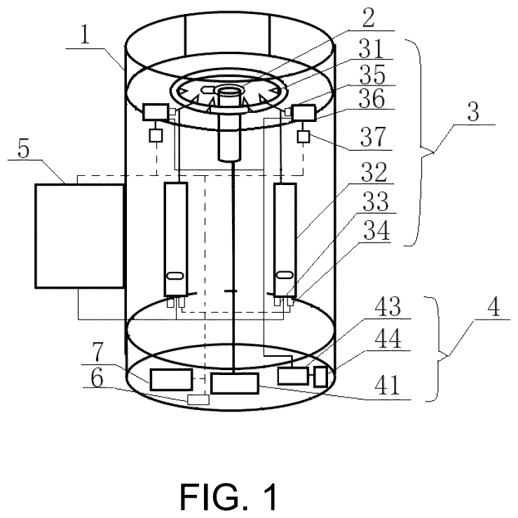

This embodiment provides an automatic pressure-retaining sampling apparatus for deep-sea specific layer seawater, as shown in FIG. 1, including an outer frame 1, a rotation unit 2, a multi-sequence sampling filter unit, an injection unit 4, a pressurization unit 5, a layer identification unit 6, and a control unit 7.

The multi-sequence sampling filter unit is disposed in the outer frame 1 and includes a plurality of sampling filter modules 3, and each of the sampling filter modules 3 includes a sampling valve 31, a sampling kettle 32, an exhaust solenoid valve 33, a pressure sensor 34, a filter valve 35, a filter kettle 36, and a flow rate meter 37; a water outlet end of the sampling valve 31 is connected to one end of the sampling kettle 32, and the other end of the sampling kettle 32 is provided with the pressure sensor 34 and the exhaust solenoid valve 33; the water outlet end of the sampling valve 31 is further connected to a water inlet end of the filter valve 35, a water outlet end of the filter valve 35 is connected to one end of the filter kettle 36, and the other end of the filter kettle 36 is connected to the flow rate meter 37; and the plurality of sampling valves 31 are circumferentially distributed at a top of the outer frame 1, and control ends of all the sampling valves 31 face the rotation unit 2.

The injection unit 4 is disposed in the outer frame 1 and includes a water pump 41, a stabilization solution injection pump 43, and a stabilization solution storage kettle 44. One end of the water pump 41 is suspended, and the other end of the water pump 41 is connected to a water inlet end of the sampling valve 31 of each of the sampling filter modules 3; and one end of the stabilization solution injection pump 43 is connected to the stabilization solution storage kettle 44, and the other end of the stabilization solution injection pump 43 is connected to one end of the filter kettle 36 of each of the sampling filter modules 3.

The rotation unit 2 is disposed at a center of the top of the outer frame 1, and an end of the rotation unit 2 abuts against the control end of the sampling valve 31.

The pressurization unit 5 is disposed outside the outer frame 1, and an output end of the pressurization unit 5 is connected to the other end of the sampling kettle 32 of each of the sampling filter modules 3.

The layer identification unit 6 is disposed at a bottom of the outer frame 1, and a data output end of the layer identification unit 6 is connected to a data input end of the control unit 7.

The control unit 7 is disposed in the outer frame 1, the data input end of the control unit 7 is further connected to data output ends of the pressure sensor 34 and the flow rate meter 37, and a control end of the control unit 7 is connected to control ends of the rotation unit 2, the pressurization unit 5, and the exhaust solenoid valve 33.

During specific implementation, with the arrangement of the layer identification unit 6, actual environmental characteristics of the seawater profile are automatically identified during the lowering process. The control unit 7 determines a plurality of target layers, calculates corresponding pre-charged pressure values, and controls the pressurization unit 5 to sequentially inject high-pressure nitrogen gas into the corresponding sampling kettles 32. When the sampling apparatus reaches the depth of a target layer during the lifting process, the rotation unit 2 rotates clockwise to open the corresponding sampling valve 31 in the multi-sequence sampling filter unit and the rotation unit 2 stop rotating when the corresponding sampling valve 31 is opened, while the control unit 7 activates the water pump 41 and opens the filter valve 35 to start sampling. After a period of sampling, the rotation unit 2 rotates clockwise to close the sampling valve 31 and the rotation unit 2 stop rotating when the sampling valve 31 is closed, and the control unit 7 deactivates the water pump 41. The pressure sensor 34 and the flow rate meter 37 respectively monitor a real-time pressure of the sampling kettle 32 and a real-time flow rate of the filter kettle 36 in real time. The control unit 7 determines whether the real-time pressure and the real-time flow rate reach preset thresholds. If both the real-time pressure and the real-time flow rate reach preset thresholds, the stabilization solution injection pump 43 is activated to stabilize the plankton in the filter kettle 36. Otherwise, the rotation unit 2 rotates counterclockwise to reopen the sampling valve 31 and the rotation unit 2 stop rotating when the sampling valve 31 is reopened, and the pressurization unit 5 is controlled to inject a pressure greater than the in-situ pressure of the target layer into the sampling kettle 32, discharging the seawater in the sampling kettle 32 through the reopened sampling valve 31. The pressure sensor 34 monitors the pressure in the sampling kettle 32 in real time, and when the real-time pressure reaches a preset pressure, it indicates that the seawater has been completely discharged. Then, the exhaust solenoid valve 33 is controlled to be opened, discharging the gas until a value of pressure in the sampling kettle 31 reaching the pre-charged value. The water pump 41 is then activated again for secondary sampling until the seawater sampling at the target layer is successful. The sampling apparatus is lifted again to the next target layer, and the above operations are repeated until the seawater sampling for all target layers is completed. According to the present invention, a specific seawater layer can be automatically selected, a sampling effect can be autonomously evaluated, and a failed sample can be automatically discharged and followed by secondary sampling, allowing for a high sampling efficiency and a good sampling effect.

### Embodiment 2

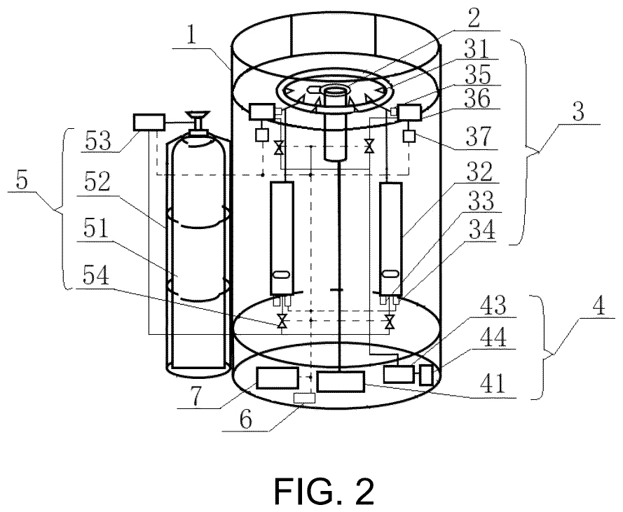

This embodiment provides an automatic pressure-retaining sampling apparatus for deep-sea specific layer seawater, as shown in FIG. 2, including an outer frame 1, a rotation unit 2, a multi-sequence sampling filter unit, an injection unit 4, a pressurization unit 5, a layer identification unit 6, and a control unit 7.

The multi-sequence sampling filter unit is disposed in the outer frame 1 and includes a plurality of sampling filter modules 3, and each of the sampling filter modules 3 includes a sampling valve 31, a sampling kettle 32, an exhaust solenoid valve 33, a pressure sensor 34, a filter valve 35, a filter kettle 36, and a flow rate meter 37; a water outlet end of the sampling valve 31 is connected to one end of the sampling kettle 32, and the other end of the sampling kettle 32 is provided with the pressure sensor 34 and the exhaust solenoid valve 33; the water outlet end of the sampling valve 31 is further connected to a water inlet end of the filter valve 35, a water outlet end of the filter valve 35 is connected to one end of the filter kettle 36, and the other end of the filter kettle 36 is connected to the flow rate meter 37; and the plurality of sampling valves 31 are circumferentially distributed at a top of the outer frame 1, and control ends of all the sampling valves 31 face the rotation unit 2.

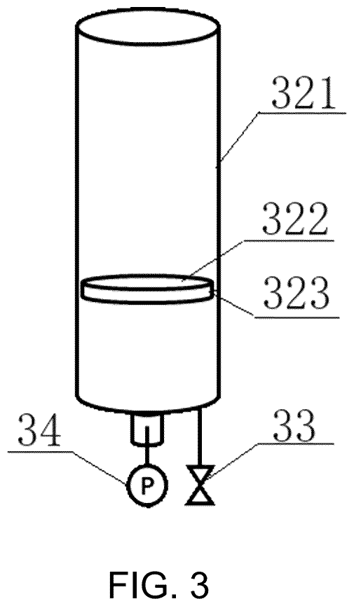

As shown in FIG. 3, the sampling kettle 32 includes a first kettle body 321, a piston 322, and a rubber ring 323.

A sidewall of the piston 322 is provided with a groove, and the rubber ring 323 is sleeved in the groove of the piston 322.

The piston 322 is disposed in the first kettle body 321 and divides the first kettle body 321 into a gas chamber and a liquid chamber.

one end of the liquid chamber of the first kettle body 321 is connected to the water outlet end of the sampling valve 31, and one end of the gas chamber of the first kettle body 321 is provided with the pressure sensor 34 and the exhaust solenoid valve 33.

The sampling kettle 32 is made of high-pressure-resistant metal material and the internally mounted piston 322 is a pressure-resistant piston 322. The sidewall is of a groove structure for mounting the rubber ring 323. The first kettle body 321 is divided by the piston 322 into the gas chamber and the liquid chamber, where the liquid chamber is used for receiving seawater samples, and the gas chamber is used for receiving high-pressure nitrogen gas. The pressure sensor 34 is configured to measure the internal pressure value of the sampling kettle 32 in real time and transmit it to the control unit 7, and the exhaust solenoid valve 33 is configured to discharge high-pressure nitrogen gas exceeding the pre-charged pressure value during secondary sampling.

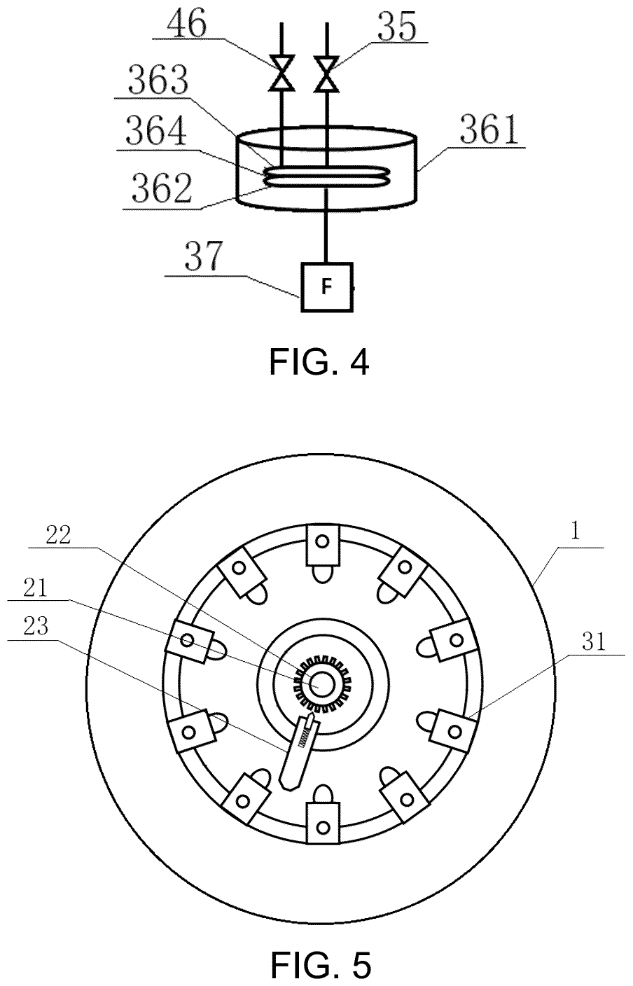

As shown in FIG. 4, the filter kettle 36 includes a second kettle body 361, a filter screen frame 362, a filter membrane 363, and a filter screen 364.

The filter screen frame 362 is disposed in the second kettle body 361, the filter screen 364 is wrapped around the filter screen frame 362, and the filter membrane 363 is disposed on the filter screen 364.

One end of the second kettle body 361 is connected to the water outlet end of the filter valve 35 and the other end of the injection valve 46, and the other end of the second kettle body 361 is connected to the flow rate meter 37.

In this embodiment, the filter screen frame 362 has multiple holes with a diameter of 5 mm, the filter screen 364 has holes with a diameter smaller than 1 mm, and the filter membrane 363 has a pore size of 0.22 μm. The filter screen 364 is configured to support the filter membrane 363. The flow rate meter 37 is configured to measure the flow rate of filtered in-situ seawater and calculate the volume of filtered in-situ seawater.

The injection unit 4 is disposed in the outer frame 1, including a water pump 41, a stabilization solution injection pump 43, a stabilization solution storage kettle 44, and a plurality of injection valves 46. One end of the water pump 41 is suspended, and the other end of the water pump 41 is connected to the water inlet end of the sampling valve 31 of each sampling filter module 3. One end of the stabilization solution injection pump 43 is connected to the stabilization solution storage kettle 44, and the other end of the stabilization solution injection pump 43 is connected to one end of each injection valve 46. The other end of each injection valve 46 is connected to one end of the filter kettle 36 of one sampling filter module 3 correspondingly. The stabilization solution storage kettle 44 contains RNAlater stabilization solution.

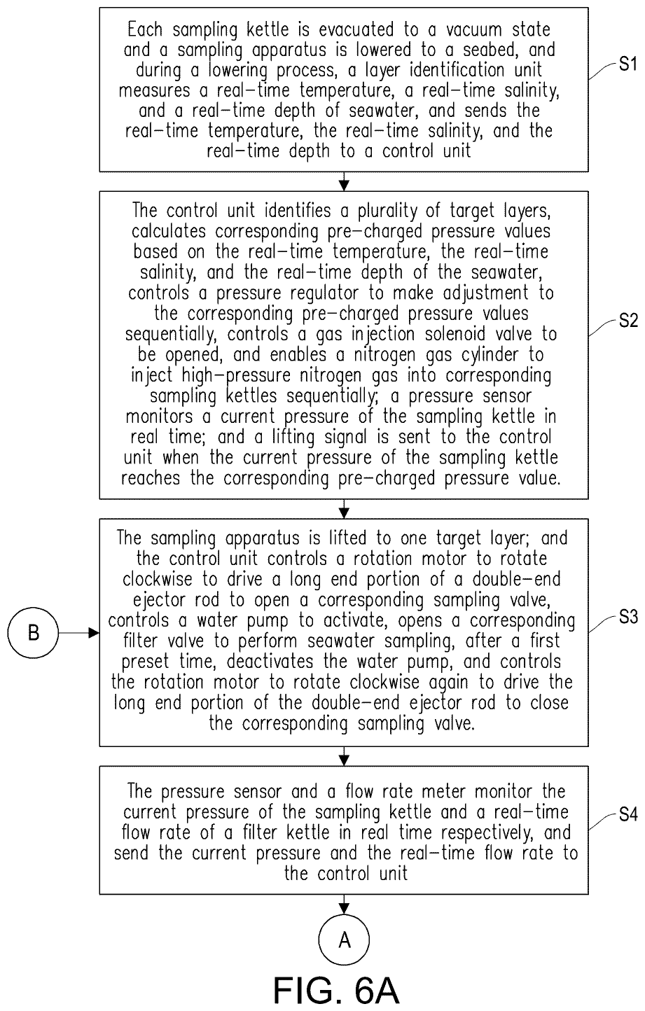

As shown in FIG. 5, the rotation unit 2 includes a rotation motor 21, a gear disc 22, and a double-end ejector rod 23.

The gear disc 22 is disposed at the center of the top of the outer frame 1.

The double-end ejector rod 23 includes a long end portion and a short end portion, the short end portion of the double-end ejector rod 23 abuts against a tooth surface of the gear disc 22, and the long end portion of the double-end ejector rod 23 abuts against the control end of the sampling valve 31.

An output end of the rotation motor 21 is connected to the double-end ejector rod 23 to drive the double-end ejector rod 23 to rotate, and a control end of the rotation motor 21 is connected to the control end of the control unit 7.

The gear disc is disposed at the center of the top of the outer frame 1, and the double-end ejector rod 23 is driven by the rotation motor 21. After rotation, the long end portion of the double-end ejector rod 23 contacts a round ejector pin of the sampling valve 31, triggering the opening and closing of the sampling valve 31. The short end portion of the double-end ejector rod 23 has a spring-loaded pin, and the spring-loaded pin, after rotation, engages in the tooth groove of the gear disc 22 for fixation. The tooth grooves of the gear disc 22 correspond to the sampling valves 31 to achieve accurate opening and closing of the sampling valves 31.

The pressurization unit 5 includes a nitrogen gas cylinder 51, a cylinder holder 52, a pressure regulator 53, and a gas injection solenoid valve 54.

The cylinder holder 52 is disposed outside the outer frame 1, and the nitrogen gas cylinder 51 is disposed in the cylinder holder 52.

A gas outlet of the nitrogen gas cylinder 51 is connected to one end of the pressure regulator 53, the other end of the pressure regulator 53 is connected to one end of the gas injection solenoid valve 54, and the other end of the gas injection solenoid valve 54 is connected to one end of the gas chamber of each of the sampling kettles 32.

The control end of the control unit 7 is connected to control ends of the pressure regulator 53 and the gas injection solenoid valve 54.

The nitrogen gas cylinder 51 is sequentially connected to the gas chamber of the sampling kettle 32 via the pressure regulator 53 and the gas injection solenoid valve 54. The pressure regulator 53 is configured to automatically adjust the pressure value of the injected nitrogen gas. The cylinder holder 52 is configured to secure the nitrogen gas cylinder 51 to prevent the cylinder from tipping over during ship operations or drifting underwater due to ocean currents and buoyancy. The cylinder holder 52 includes three layers of railings, with latches provided on the railings on the same side of the three layers, all of which can be opened outward, facilitating the transfer of the nitrogen gas cylinder 51 into the cylinder holder 52 and secure locking.

The layer identification unit 6 is disposed at the bottom of the outer frame 1, including a depth sensor 61, a temperature sensor 62, and a salinity sensor 63. The data output ends of the depth sensor 61, the temperature sensor 62, and the salinity sensor 63 are connected to the data input end of the control unit 7.

In this embodiment, the layer identification unit 6 is a CTD instrument, capable of accurately measuring the conductivity, temperature, and depth parameters of the ocean water body.

The control unit 7 is disposed in the outer frame 1, the data input end of the control unit 7 is further connected to data output ends of the pressure sensor 34 and the flow rate meter 37, and the control end of the control unit 7 is connected to the control end of the exhaust solenoid valve 33.

During specific implementation, with the arrangement of the layer identification unit 6, actual environmental characteristics of the seawater profile are automatically identified during the lowering process, and a real-time temperature, a real-time salinity, and a real-time depth of the seawater are measured and sent to the control unit. The control unit 7 determines a plurality of target layers, calculates corresponding pre-charged pressure values, and controls the pressure regulator 53 to make adjustment to the corresponding pre-charged pressure values. The gas injection solenoid valve 54 is opened, and the nitrogen gas cylinder 51 injects high-pressure nitrogen gas into the corresponding sampling kettles 32 sequentially. When the sampling apparatus reaches the depth of a target layer during the lifting process, the rotation motor 21 drives the double-end ejector rod 23 to rotate clockwise, with the short end portion engaging in the tooth groove of the gear disc 22 for stabilization, and the long end portion opening the corresponding sampling valve 31 in the multi-sequence sampling filter unit, while the control unit 7 activates the water pump 41 and open the filter valve 35 to start sampling. After a period of sampling, the rotation motor 21 drives the double-end ejector rod 23 to rotate clockwise, with the short end portion disengaging from the tooth groove of the gear disc 22, and the long end portion closing the sampling valve 31, while the control unit 7 deactivates the water pump 41. The pressure sensor 34 and the flow rate meter 37 respectively monitor the real-time pressure of the sampling kettle 32 and the real-time flow rate of the filter kettle 36 in real time. The control unit 7 determines whether the real-time pressure and the real-time flow rate reach preset thresholds. If both the real-time pressure and the real-time flow rate reach preset thresholds, the stabilization solution injection pump 43 is activated to stabilize the plankton in the filter kettle 36. Otherwise, the rotation motor 21 drives the double-end ejector rod 23 to rotate counterclockwise to reopen the sampling valve 31, the control unit 7 controls the pressure regulator 53 to make adjustment to a pressure greater than the in-situ pressure of the target layer, and the nitrogen gas cylinder 51 injects this pressure into the corresponding sampling kettle 32, discharging the seawater in the sampling kettle 32 via the sampling valve 31. The pressure sensor 34 monitors the pressure in the sampling kettle 32 in real time, and when the real-time pressure reaches a preset pressure, it indicates that the seawater has been completely discharged. Then, the exhaust solenoid valve 33 is controlled to be opened, discharging the gas until a value of pressure in the sampling kettle reaching the pre-charged value. The water pump 41 is then activated again for secondary sampling until the seawater sampling at the target layer is successful. The sampling apparatus is lifted again to the next target layer, and the above operations are repeated until the seawater sampling for all target layers is completed. According to the present invention, a specific seawater layer can be automatically selected, a sampling effect can be autonomously evaluated, and a failed sample can be automatically discharged and followed by secondary sampling, allowing for a high sampling efficiency and a good sampling effect.

### Embodiment 3

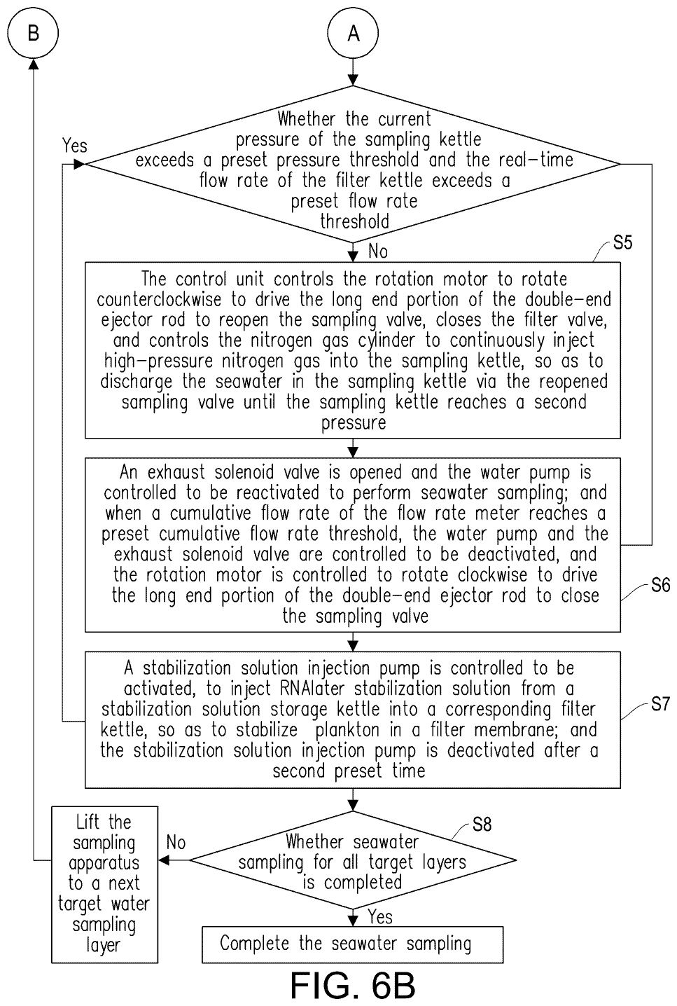

This embodiment provides an automatic pressure-retaining sampling method for deep-sea specific layer seawater, applied to the sampling apparatus described in Embodiment 1 or 2, and as shown in FIG. 6A and FIG. 6B, including:
S1: evacuating each sampling kettle to a vacuum state and lowering the sampling apparatus to a seabed; and during a lowering process, measuring, by a layer identification unit, a real-time temperature, a real-time salinity, and a real-time depth of the seawater, and sending the real-time temperature, the real-time salinity, and the real-time depth to a control unit;
S2: identifying, by the control unit, a plurality of target layers, calculating corresponding pre-charged pressure values based on the real-time temperature, the real-time salinity, and the real-time depth of the seawater, controlling a pressure regulator to make adjustment to the corresponding pre-charged pressure values sequentially, controlling a gas injection solenoid valve to be opened, and enabling a nitrogen gas cylinder to inject high-pressure nitrogen gas into corresponding sampling kettles sequentially; monitoring, by a pressure sensor, a current pressure of the sampling kettle in real time; and sending a lifting signal to the control unit when the current pressure of the sampling kettle reaches the corresponding pre-charged pressure value;
S3: lifting the sampling apparatus to one target layer; and controlling, by the control unit, a rotation motor to rotate clockwise to drive a long end portion of a double-end ejector rod to open a corresponding sampling valve and the rotation motor to stop rotate when the corresponding sampling valve is opened, controlling a water pump to be activated, opening a corresponding filter valve to perform seawater sampling, after a first preset time, deactivating the water pump, and controlling the rotation motor to rotate clockwise to drive the long end portion of the double-end ejector rod to close the corresponding sampling valve and the rotation motor to stop rotate when the corresponding sampling valve is closed;
S4: monitoring, by the pressure sensor and a flow rate meter, the current pressure of the sampling kettle and a real-time flow rate of a filter kettle in real time respectively, and sending the current pressure and the real-time flow rate to the control unit; and performing step S7 when the current pressure of the sampling kettle exceeds a preset pressure threshold and the real-time flow rate of the filter kettle exceeds a preset flow rate threshold; otherwise, performing step S5;
S5: controlling, by the control unit, the rotation motor to rotate counterclockwise to drive the long end portion of the double-end ejector rod to reopen the sampling valve and the rotation motor to stop rotate when the sampling valve is reopened, closing the filter valve, and controlling the nitrogen gas cylinder to continuously inject high-pressure nitrogen gas into the sampling kettle, so as to discharge the seawater in the sampling kettle via the reopened sampling valve until the sampling kettle reaches a second pressure;
S6: opening an exhaust solenoid valve and controlling the water pump to reactivate to perform seawater sampling; and when a cumulative flow rate of the flow rate meter reaches a preset cumulative flow rate threshold, controlling the water pump and the exhaust solenoid valve to be deactivated, and controlling the rotation motor to rotate clockwise to drive the long end portion of the double-end ejector rod to close the sampling valve and the rotation motor to stop rotate when the sampling valve is closed; and returning to step S4;
S7: controlling a stabilization solution injection pump to be activated, to inject RNAlater stabilization solution from a stabilization solution storage kettle into a corresponding filter kettle, so as to stabilize plankton in a filter membrane; and deactivating the stabilization solution injection pump after a second preset time; and
S8: determining whether seawater sampling for all target layers is completed; and if the seawater sampling is not completed, lifting the sampling apparatus to a next target water sampling layer and repeating steps S3 to S7; otherwise, terminating the seawater sampling.

The identifying, by the control unit, the plurality of target layers and calculating corresponding pre-charged pressure values based on the real-time temperature, the real-time salinity, and the real-time depth of the seawater includes:
using a plurality of seawater layers, each having a variation value of the real-time temperature or the real-time salinity of the seawater exceeding a preset variation threshold, as the target layers, and calculating the corresponding pre-charged pressure values of the target layers based on the corresponding real-time depths:

$${N}_{i} = \frac{( V - {V}_{L} ) {D}_{i}}{100 \times V}$$

where Nᵢ represents a pre-charged pressure value corresponding to an i-th target layer, Dᵢ represents a real-time depth of the i-th target layer, V represents a volume of the sampling kettle, and V_{L} represents an expected volume after in-situ pressure compression of the sampling kettle at the i-th target layer. In this embodiment, the volume V of the sampling kettle is 5 liters, and V_{L} is 4.5 liters.

In this embodiment, the preset pressure threshold is Dᵢ/100 megapascals, the preset flow rate threshold is 4500 cubic centimeters, and the second pressure is 5+Dᵢ/100 megapascals.

Identical or similar reference numerals correspond to identical or similar components.

Terms describing positional relationships in the drawings are for illustrative purposes only and should not be construed as limiting this patent.

Obviously, the above embodiments of the present invention are merely examples to clearly illustrate the present invention and are not intended to limit the implementations of the present invention. For those of ordinary skill in the art, other variations or modifications in different forms can be made based on the above description. It is neither necessary nor possible to exhaustively list all implementations herein. Any modifications, equivalent substitutions, improvements, and the like made within the spirit and principles of the present invention shall be included within the scope of protection of the claims of the present invention.

What is claimed is:

1. An automatic pressure-retaining sampling method for deep-sea specific layer seawater, applied to an automatic pressure-retaining sampling apparatus for deep-sea specific layer seawater, and comprising:
S1: evacuating each sampling kettle to a vacuum state and lowering the sampling apparatus to a seabed; and during a lowering process, measuring, by a layer identification unit, a real-time temperature, a real-time salinity, and a real-time depth of the seawater, and sending the real-time temperature, the real-time salinity, and the real-time depth to a control unit;
S2: identifying, by the control unit, a plurality of target layers, calculating corresponding pre-charged pressure values based on the real-time temperature, the real-time salinity, and the real-time depth of the seawater, controlling a pressure regulator to make adjustment to the corresponding pre-charged pressure values sequentially, controlling a gas injection solenoid valve to be opened, and enabling a nitrogen gas cylinder to inject high-pressure nitrogen gas into corresponding sampling kettles sequentially; monitoring, by a pressure sensor, a current pressure of the sampling kettle in real time; and sending a lifting signal to the control unit when the current pressure of the sampling kettle reaches the corresponding pre-charged pressure value;
S3: lifting the sampling apparatus to a target layer; and controlling, by the control unit, a rotation motor to rotate clockwise to drive a long end portion of a double-end ejector rod to open a corresponding sampling valve, controlling a water pump to be activated, opening a corresponding filter valve to perform seawater sampling, after a first preset time, deactivating the water pump, and controlling the rotation motor to rotate clockwise to drive the long end portion of the double-end ejector rod to close the corresponding sampling valve;
S4: monitoring, by the pressure sensor and a flow rate meter, the current pressure of the sampling kettle and a real-time flow rate of a filter kettle in real time respectively, and sending the current pressure and the real-time flow rate to the control unit; and performing step S7 when the current pressure of the sampling kettle exceeds a preset pressure threshold and the real-time flow rate of the filter kettle exceeds a preset flow rate threshold; otherwise, performing step S5;
S5: controlling, by the control unit, the rotation motor to rotate counterclockwise to drive the long end portion of the double-end ejector rod to reopen the sampling valve, closing the filter valve, and controlling the nitrogen gas cylinder to continuously inject high-pressure nitrogen gas into the sampling kettle, so as to discharge the seawater in the sampling kettle via the reopened sampling valve until the sampling kettle reaches a second pressure;
S6: opening an exhaust solenoid valve and controlling the water pump to be reactivated to perform seawater sampling; and when a cumulative flow rate of the flow rate meter reaches a preset cumulative flow rate threshold, controlling the water pump and the exhaust solenoid valve to be deactivated, and controlling the rotation motor to rotate clockwise to drive the long end portion of the double-end ejector rod to close the sampling valve; and returning to step S4;
S7: controlling a stabilization solution injection pump to be activated, to inject RNAlater stabilization solution from a stabilization solution storage kettle into a corresponding filter kettle, so as to stabilize plankton in a filter membrane; and deactivating the stabilization solution injection pump after a second preset time; and
S8: determining whether seawater sampling for all target layers is completed; and if the seawater sampling is not completed, lifting the sampling apparatus to a next target water sampling layer and repeating steps S3 to S7; otherwise, terminating the seawater sampling; wherein
the automatic pressure-retaining sampling apparatus for deep-sea specific layer seawater comprises an outer frame, a rotation unit, a multi-sequence sampling filter unit, an injection unit, a pressurization unit, the layer identification unit, and the control unit; wherein
the multi-sequence sampling filter unit is disposed in the outer frame and comprises a plurality of sampling filter modules, and each of the sampling filter modules comprises the sampling valve, the sampling kettle, the exhaust solenoid valve, the pressure sensor, the filter valve, the filter kettle, and the flow rate meter; a water outlet end of the sampling valve is connected to one end of the sampling kettle, and the other end of the sampling kettle is provided with the pressure sensor and the exhaust solenoid valve; the water outlet end of the sampling valve is further connected to a water inlet end of the filter valve, a water outlet end of the filter valve is connected to one end of the filter kettle, and the other end of the filter kettle is connected to the flow rate meter; and the plurality of sampling valves are circumferentially distributed at a top of the outer frame, and control ends of all the sampling valves face the rotation unit;
the injection unit is disposed in the outer frame and comprises the water pump, the stabilization solution injection pump, and the stabilization solution storage kettle; wherein one end of the water pump is suspended, and the other end of the water pump is connected to a water inlet end of the sampling valve of each of the sampling filter modules; and one end of the stabilization solution injection pump is connected to the stabilization solution storage kettle, and the other end of the stabilization solution injection pump is connected to one end of the filter kettle of each of the sampling filter modules;
the rotation unit is disposed at a center of the top of the outer frame, and an end of the rotation unit abuts against the control end of the sampling valve;
the pressurization unit is disposed outside the outer frame, and an output end of the pressurization unit is connected to the other end of the sampling kettle of each of the sampling filter modules;
the layer identification unit is disposed at a bottom of the outer frame, and a data output end of the layer identification unit is connected to a data input end of the control unit;
the control unit is disposed in the outer frame, the data input end of the control unit is further connected to data output ends of the pressure sensor and the flow rate meter, and a control end of the control unit is connected to control ends of the rotation unit, the pressurization unit, and the exhaust solenoid valve;
the sampling kettle comprises a first kettle body, a piston, and a rubber ring;
a sidewall of the piston is provided with a groove, and the rubber ring is sleeved in the groove of the piston;
the piston is disposed in the first kettle body and divides the first kettle body into a gas chamber and a liquid chamber;
an end of the liquid chamber of the first kettle body is connected to the water outlet end of the sampling valve, and an end of the gas chamber of the first kettle body is provided with the pressure sensor and the exhaust solenoid valve;
the pressurization unit comprises the nitrogen gas cylinder, a cylinder holder, the pressure regulator, and the gas injection solenoid valve;
the cylinder holder is disposed outside the outer frame, and the nitrogen gas cylinder is disposed in the cylinder holder;
a gas outlet of the nitrogen gas cylinder is connected to one end of the pressure regulator, the other end of the pressure regulator is connected to one end of the gas injection solenoid valve, and the other end of the gas injection solenoid valve is connected to one end of the gas chamber of each of the sampling kettles;
the control end of the control unit is connected to control ends of the pressure regulator and the gas injection solenoid valve;
the rotation unit comprises the rotation motor, a gear disc, and the double-end ejector rod;
the gear disc is disposed at the center of the top of the outer frame;
the double-end ejector rod comprises a long end portion and a short end portion, the short end portion of the double-end ejector rod abuts against a tooth surface of the gear disc, and the long end portion of the double-end ejector rod abuts against the control end of the sampling valve; and
an output end of the rotation motor is connected to the double-end ejector rod to drive the double-end ejector rod to rotate, and a control end of the rotation motor is connected to the control end of the control unit.

2. The automatic pressure-retaining sampling method for deep-sea specific layer seawater according to claim 1, wherein the layer identification unit comprises a depth sensor, a temperature sensor, and a salinity sensor; and
the depth sensor, the temperature sensor, and the salinity sensor are all disposed at the bottom of the outer frame, and data output ends of the depth sensor, the temperature sensor, and the salinity sensor are all connected to the data input end of the control unit.

3. The automatic pressure-retaining sampling method for deep-sea specific layer seawater according to claim 1, wherein the injection unit further comprises a plurality of injection valves; and
the other end of the stabilization solution injection pump is connected to one end of each of the injection valves, and the other end of each of the injection valves is connected to one end of the filter kettle of one sampling filter module correspondingly.

4. The automatic pressure-retaining sampling method for deep-sea specific layer seawater according to claim 3, wherein the filter kettle comprises a second kettle body, a filter screen frame, the filter membrane, and a filter screen;
the filter screen frame is disposed in the second kettle body, the filter screen is wrapped around the filter screen frame, and the filter membrane is disposed on the filter screen; and
one end of the second kettle body is connected to the water outlet end of the filter valve and the other end of the injection valve, and the other end of the second kettle body is connected to the flow rate meter.

5. The automatic pressure-retaining sampling method for deep-sea specific layer seawater according to claim 1, wherein the stabilization solution storage kettle contains the RNAlater stabilization solution.

6. The automatic pressure-retaining sampling method for deep-sea specific layer seawater according to claim 1, wherein the identifying, by the control unit, the plurality of target layers and calculating the corresponding pre-charged pressure values based on the real-time temperature, the real-time salinity, and the real-time depth of the seawater comprises:
using a plurality of seawater layers, each having a variation value of the real-time temperature or the real-time salinity of the seawater exceeding a preset variation threshold, as the target layers, and calculating the corresponding pre-charged pressure values of the target layers based on the corresponding real-time depths:
$${N}_{i} = \frac{( V - {V}_{L} ) {D}_{i}}{100 \times V}$$
wherein Nᵢ represents a pre-charged pressure value corresponding to an i-th target layer, Dᵢ represents a real-time depth of the i-th target layer, V represents a volume of the sampling kettle, and V_{L} represents an expected volume after in-situ pressure compression of the sampling kettle at the i-th target layer.
